(19) United States
(12) Patent Application Publication
(10) Pub. No.: US 2020/0278486 A1
(43) Pub. Date: Sep. 3, 2020

(54) ILLUMINATION SYSTEM

(71) Applicants: Eduardo SVETLIZA, CAESAREA (IL); Yigal DAHAN, CAESAREA (IL); Oded ANNER, CAESAREA (IL)

(72) Inventors: Eduardo SVETLIZA, CAESAREA (IL); Yigal DAHAN, CAESAREA (IL); Oded ANNER, CAESAREA (IL)

(21) Appl. No.: 16/878,553

(22) Filed: May 19, 2020

Related U.S. Application Data
(62) Division of application No. 15/758,606, filed on Mar. 8, 2018.
(60) Provisional application No. 62/292,367, filed on Feb. 8, 2016.

(51) Int. Cl. F21V 8/00; G02B 6/28; A61B 3/12; A61B 3/00

(52) U.S. Cl. CPC G02B 6/0008; G02B 6/0006; A61B 3/0008; A61B 3/12; G02B 6/28

(57) ABSTRACT

An integrated ophthalmic illumination system comprising: (1) a circumferential ring, having a tangential cross-section, (2) at least one light source comprised of a light beam of multiple wavelengths, (3) a mediating mixing element, the mediating mixing element placed in proximity to the at least one light source to receive and to transform the light beam into a mixed beam, (4) a plurality of light guiding elements, the plurality of light guiding elements placed in close proximity to the output of the mediating mixing element to receive and to convey the mixed beam to the circumferential ring, and (5) a controller connected to the at least one light source for controlling light intensity, light distribution, and restricted light of predetermined wavelengths.

### FIELD OF THE INVENTION

The present invention relates to an ophthalmic illumination system. More particularly, the present invention relates to a stand-alone, wide angle, diffuse ophthalmic illumination system for illuminating the interior of the eye during examinations, treatments and surgeries.

### BACKGROUND OF THE INVENTION

There is an increasing need in accordance with a preferred embodiment of the present invention, and specifically in ophthalmic applications for compact, efficient, modular and broad band high brightness illumination systems. While various illumination systems and methods have been proposed in the past, they all use a lamp as a light. Relevant prior-art references using filament based or short arc lamps such as halogen, metal halide, high pressure mercury and xenon are disclosed below:

U.S. Pat. No. 3,954,329 describes apparatus for viewing an eye fundus through a contact lens. The apparatus has a lamp element that illuminates the fundus through the sclera.

U.S. Pat. No. 4,023,189 discloses a wide angle instrument for illuminating, observing and photographing the fundus of the eye. The instrument utilizes an arc-lamp and has a focus tube containing spaced decollimating and objective lenses with an adjustable aperture diaphragm positioned therebetween.

U.S. Pat. No. 5,822,036 describes an eye imaging system having a portable image capture unit having a circular light guide positioned adjacent to and behind a corneal contact lens for controlling directing lamp light over a wide field to the retina of an eye and provide more light towards the center of the eye.

US20070030448 is directed to an optical device for the observation and documentation of the ocular fundus and is preferably provided for fundus cameras. In order to generate a uniform illumination of the fundus by trans illumination of the sclera in the illumination unit, for fundus cameras and/or ophthalmoscopes, the light emitted by the illumination source, such as a lamp, is coupled into individual light-conducting fibers or bundles of light-conducting fibers which extend into the area of the front lens of the fundus camera and ophthalmoscope and whose fiber ends are formed in such a way that the exiting light is projected on and trans illuminates the sclera.

U.S. Pat. No. 6,309,070 of Eduardo Svetliza, the inventor of the present invention discloses an integrated ophthalmic illumination method and system having two integrated light sources, a lamp and an infra-red (IR) diode laser. The lamp light source may be used to produce either monochromatic or color images, as necessary, at high resolution.

The problems involved with usage of lamps include poor luminous efficacy, high power and cooling requirements, environmental and user hazards and short lifetimes. A typical multi-color system using such lamps requires a set of filters and optics to separate the spectrum of the light produced by such lamps to the desired spectral components. In addition, the system will usually require a fast shutter due to the slow activation and slow deactivation of such lamps.

The above drawbacks are overcome when replacing the lamp with Light Emitting Diodes (LEDs). The technology of LEDs is rapidly growing and gradually replacing all current forms of ambient illumination specifically incandescent and fluorescent based light bulbs. With daily improvements in efficiency and power output, LEDs have the potential to replace all traditional light sources with the added benefits of very long lifetimes, low cost, lower power consumption, low voltage operation, simple cooling requirements and very rapid power output modulation (typically microseconds on-off times). LEDs are available as monochromatic sources (from the UV to the NIR spectrum) or as a more broadband source when combined with phosphors deposited on the LED emitter.

Thus, LEDs are an ideal light source for ophthalmic applications, enabling simple power and spectral output control in a compact package with a very long lifetime. The following prior-art references describe LED-based illumination systems:

U.S. Pat. No. 5,695,492 discloses apparatus for illuminating a central area of an eye by generally lamellar lighting during eye surgery. Basically, a support fixture carrying a light emitter such as a LED is adapted to be placed adjacent to the surgical field. The support fixture, when in place on an eye, directs light from the light emitter toward the surgical field tangentially to the cornea, at an angle of from about 0 degrees to 90 degrees to the plane of the eye iris. The light entering the eye travels along the lamellae of the cornea in the manner of a light pipe. Very little, if any light reaches the back of the eye, avoiding patient discomfort, or is directed toward the surgical microscope as glare.

US 20100318074 discloses an ophthalmic surgical system which includes a laser light source having a laser treatment mode and an illumination mode. The illumination system comprises a handpiece which is inserted into the eye through an incision in the pars plana region to illuminate the inside or vitreous region of the eye. Handpiece is connected to a laser light source by a light guide which is typically an optical fiber.

U.S. Pat. No. 5,966,196 of Eduardo Svetliza, the inventor of the present invention discloses apparatus for wide angle examination of the eye fundus. The apparatus includes an optical module providing a wide angle view image of the eye fundus and an image capturing unit connected to the optical module for capturing the wide angle view image. The apparatus also includes an illumination system comprising LEDs connected to a plurality of light guiding elements which are capable of transferring light from the LEDs to the eye.

It is an aim of the present invention to provide an integrated illumination system of low cost that is safe, easy to operate, and precise in any ophthalmic eye retina applications.

It is another aim of the present invention to provide an illumination system that is significantly small and compact, portable, and cordless to allow easy access to treated or monitored locations.

It is yet another aim of the present invention to provide an illumination system for controlling restricted light penetration and for superb manipulation of light and the resulting image.

### SUMMARY OF THE INVENTION

A solid state based illumination system, in accordance with the present invention, illuminates the fundus through the sclera via direct contact or in very close proximity of the illumination system to the sclera.

The illumination system in accordance with the present invention provides a complete control of the light sources, i.e., control over parameters such as the light wavelengths and illuminating angle of projection light into the cavity of the eye as desired by the ophthalmologist.

The illumination system of the present invention comprises lighting elements required for retinal diagnosis such as perfect balanced color imaging, monochromatic restricted light imaging, and fluorescein angiography (FA) and Indocyanine green (ICG) in a single light source.

The illumination system of the present invention is based on a portable, cordless, small, compact and efficient LED ring with no fiber mediation for guiding light from one point to another and with minimal voltage/current requirements. Due to such characteristics, the LED ring of the present invention is a stand-alone ring that may be operated by a battery. Moreover, the LED ring is relatively small and compact to allow easy access to treated or monitored locations. This saves space and minimizes losses.

Additional advantages of the LED ring of the present invention are listed as follows:

1. The LED ring is designed to provide several modes of illumination. According to one mode of illumination, all of the LEDs are turned on as to provide an even illumination of the examined eye fundus. According to another mode of illumination, a selected group of LEDs is turned on while the rest of the LEDs are turned off, thereby illuminating the eye from a selected angle. For instance, an illumination angle of up to 270 degrees may be used in retina lighting surgery such as vitrectomy. Since such illumination angle may provide the required illumination for the surgery, insertion of a light probe thru the sclera may be avoided.

2. The LED ring may be used for angiography (fluorescein angiography-FA or indocyanine angiography-ICG) by using LEDs at the appropriate excitation wavelengths.

3. The LED ring may be used as a retractor of eyelids via direct scleral contact.

In accordance with some embodiments of the present invention, there is provided an integrated ophthalmic illumination system comprising:

a circumferential ring, having a tangential cross-section,

at least one miniature light source, said at least one miniature light source being mounted on the periphery of said circumferential ring, the light output of said at least one miniature light source is aimed at and illuminates the eye directly through the eye globe, and

a controller connected to said at least one miniature light source for controlling light intensity, light distribution, and restricted light of predetermined wavelengths.

wherein said circumferential ring is placed in the vicinity of the eye as a result of which said at least one miniature light source is either in close proximity to the eye or in contact with the eye during operation,

thereby said illumination system undergoing minimal light losses and having minimal voltage/current requirements.

In accordance with some embodiments of the present invention, there is also provided

An integrated ophthalmic illumination system comprising:

a circumferential ring, having a tangential cross-section,

at least one light source comprised of a light beam of multiple wavelengths,

a mediating mixing element, said mediating mixing element placed in proximity to said at least one light source to receive and to transform said light beam into a mixed beam,

a plurality of light guiding elements, said plurality of light guiding elements placed in close proximity to the output of said mediating mixing element to receive and to convey said mixed beam to said circumferential ring, and

a controller connected to said at least one light source for controlling light intensity, light distribution, and restricted light of predetermined wavelengths.

Furthermore, in accordance with the present invention, the light source is a solid state light source (SSLS) selected from LEDs, diode lasers, or diode pumped solid state lasers.

Furthermore, in accordance with the present invention, each one of said at least one miniature light source comprising a micro lens to collimate and direct the light into the eye.

Furthermore, in accordance with the present invention, each one of said at least one miniature light source comprising an annular window contacting the eye.

Furthermore, in accordance with the present invention, each one of said at least one miniature light source comprised of a micro lens collimating and directing the light into the eye.

Furthermore, in accordance with the present invention, said circumferential ring connected to a temperature detection element.

Furthermore, in accordance with the present invention, a band pass filter is placed against said at least one miniature light source.

Furthermore, in accordance with the present invention, said circumferential ring comprising between 1 to 18 light sources.

Furthermore, in accordance with the present invention, said controller operating said at least one light source either in parallel or in series.

Furthermore, in accordance with the present invention, said controller enabling separate control of each one of the at least one light source.

Furthermore, in accordance with the present invention, said controller monitoring the electrical power injected to each one of said at least one light source.

Furthermore, in accordance with the present invention, said controller monitoring the optical output each one of said at least one light source.

Furthermore, in accordance with the present invention, said illumination system is activated either via voice, pedals or manually.

Furthermore, in accordance with the present invention, said mediating mixing element comprised of a compound concentrator.

Furthermore, in accordance with the present invention, said mediating mixing element comprised of at least one mixing rod.

Furthermore, in accordance with the present invention, said mediating mixing element comprised of two mixing rods forming a Y shaped configuration.

Furthermore, in accordance with the present invention, said mediating mixing element comprised of a compound concentrator and at least one mixing rod.

### BRIEF DESCRIPTION OF THE DRAWINGS

For a better understanding of the invention with regard to the embodiments thereof, reference is made to the accompanying drawings in which like numerals designate corresponding elements or sections throughout and in which:

FIGS. 1A-C illustrate illumination systems in accordance with some embodiments of the present invention;

FIGS. 2A-C illustrate additional illumination systems in accordance with some embodiments of the present invention;

FIG. 3A-C illustrate further illumination systems in accordance with some embodiments of the present invention;

FIG. 4 illustrates control means for controlling any one of the illumination systems described above;

FIG. 5 illustrates fiber optic cables distributed around the sclera;

FIG. 6 shows mixing rod in accordance with some embodiments of the present invention;

FIG. 7 illustrates a compound parabolic concentrator (CPC) in accordance with some embodiments of the present invention;

FIG. 8 illustrates mixing device in accordance with some embodiments of the present invention.

FIG. 9 illustrates another mixing device in accordance with some embodiments of the present invention.

FIG. 10A shows a cross sectional view of a LED ring in accordance with some embodiments of the present invention.

FIG. 10B shows the LED ring of FIG. 10A in contact with a sclera

FIG. 11 illustrates top view of a LED ring with 12 LEDs in accordance with some embodiments of the present invention.

### DETAILED DESCRIPTION OF THE DRAWINGS

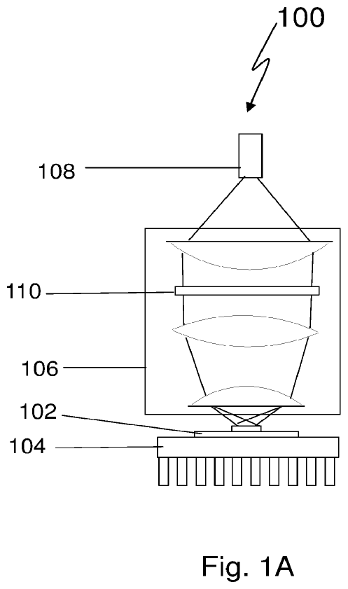

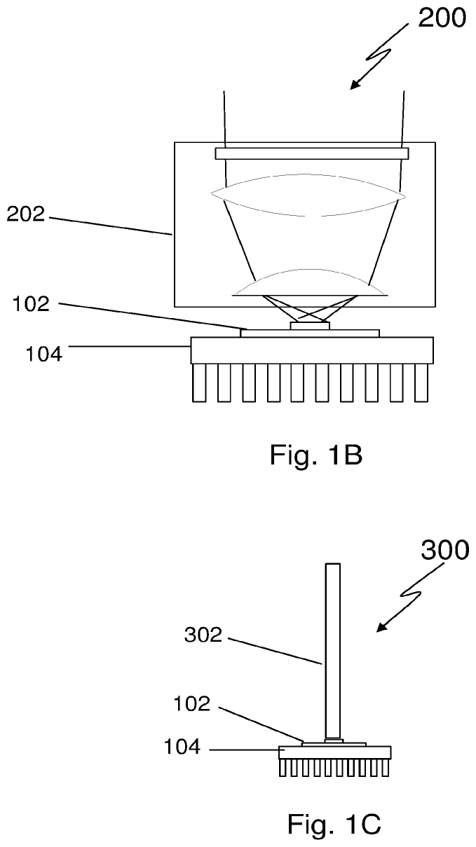

FIGS. 1A-C, illustrate illumination systems 100, 200, and 300 in accordance with some embodiments of the present invention.

Referring now to FIG. 1A, illumination system 100 includes the following: a light source 102, cooling device 104, optical system 106, light guiding element (fiber optic cable) 108, and band pass filter 110.

In accordance with some embodiments of the present invention, light source 102, is selected from solid state light source (SSLS) such as LEDs, diode lasers, diode pumped solid state lasers or a combination of such. Light source 102 provides a set of illumination colors required for diagnosis, treatment, or surgery in certain medical applications and specifically in ophthalmic applications.

Optical system 106 is positioned against light source 102 to receive the light source respective output, collimate the light and couple it to a fiber optic cable 108.

Optical system 106 may comprise multiple lenses that may be spherical, aspheric, cylindrical or of any other shape made of glass, plastic or optical ceramic. Optical system 106 may also comprise a parabolic concentrator.

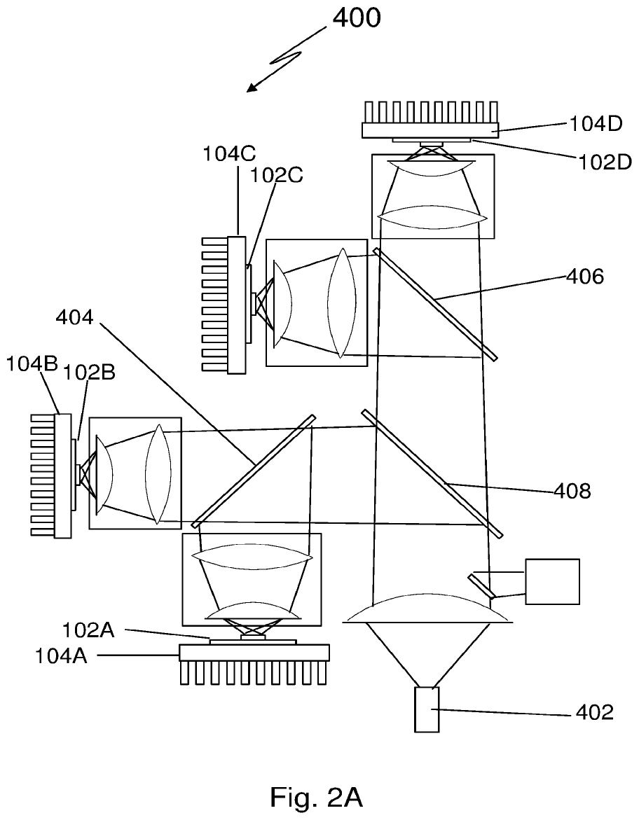

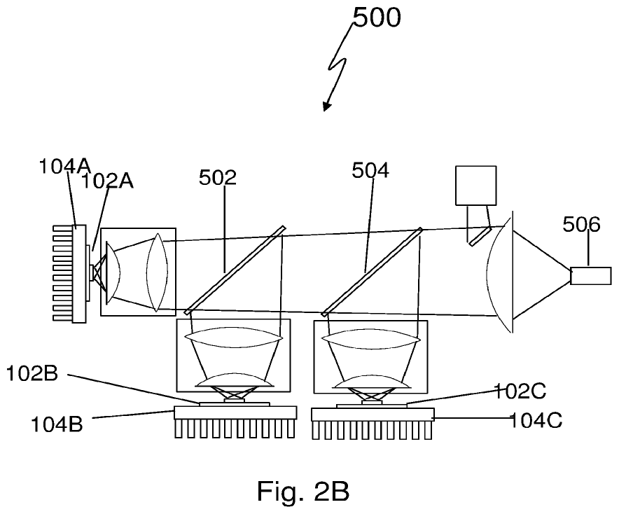

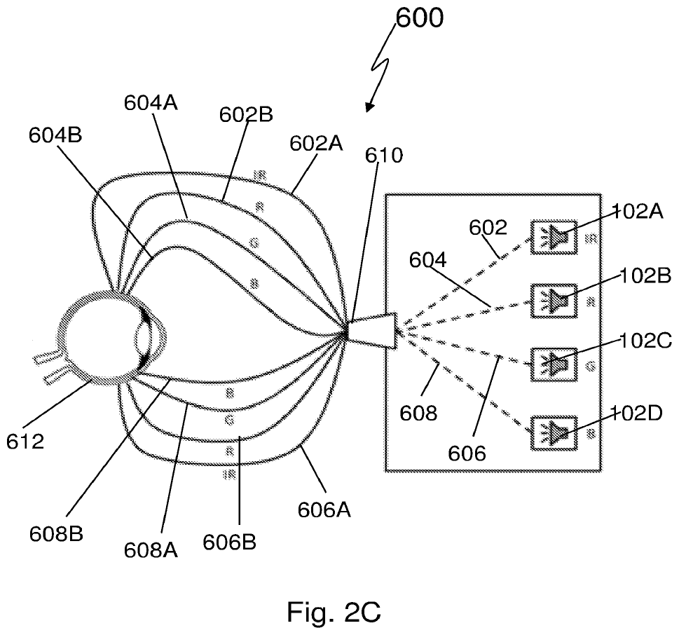

In accordance with some embodiments of the present invention, optical system 106 is able to extract high intensity light from light source 102 and collimate it to the level required by the dichroic beam combiner (shown and described in FIG. 2A) for best reflectance, transmittance and minimum losses.

Cooling device 104, in accordance with some embodiments of the present invention, may be a simple heat conducting plate, a finned heat sink, a heat sink integrated with a fan, a heat sink integrated with thermoelectric cooling device, a heat sink integrated with heat pipes, a water cooled heat sink or any other suitable cooling system.

Band pass filter 110 defines spectral band/s received from light source 102. Band pass filter 110 may define spectral band/s received from multiple light sources.

Fiber optic cable 108 may be selected from fiber optic cables, liquid light guide cables, and the like.

Referring now to FIG. 1B, illumination system 200 includes the following:

a light source 102, cooling device 104, and optical system 202 which is a collimating optical system required for operation with a dichroic beam combiner.

Referring now to FIG. 1C, illumination system 300 includes the following: light source 102, cooling device 104, and fiber optic cable 302. In this case, the output from light source 102 is extracted directly to fiber optic cable 302 with no mediating optics.

The desired shape of the light output from fiber optic cable 302 is achieved by transforming the geometry of fiber optic cable 302 and by adding optional optical components which alter the shape of the light output from fiber optic cable 302.

Illumination systems 100, 200 and 300 further include a power monitoring system (not shown in the figures) controlling and providing indication of input power to each light source or to a combination of multiple light sources.

Referring now to FIGS. 2A-C, there are shown illumination systems 400, 500, and 600 in accordance with some embodiments of the present invention.

In FIG. 2A illumination system 400 comprising 4 light sources 102A, 102B, 102C and 102D each of which is positioned on cooling platforms 104A, 1046, 104C and 104D respectively. As seen in the figure, the output from four light sources 102A, 1026, 102C and 102D are combined to a single output which passes through fiber optic cable 402. Light sources 102A, 1026, 102C and 102D initially radiate on separate optical axes and are then combined via dichroic beam combiners 404 406 and 408 to a single multi-colored optical beam.

Dichroic beam combiner 406 combines the output of light sources 102C and 102D, dichroic beam combiner 404 combines the output of light sources 102A and 1026, and dichroic beam combiner 408 combines the output of combined light sources 102A and 1026 with the output of combined light sources 102C and 102D. The combined output exiting from beam combiner 408 is fed to fiber optic cable 402 from which the various colored light beams are emitted homogeneously.

In FIG. 2B illumination system 500 includes 2 dichloric combiners to combine the light output from 3 light sources into a single beam. Illumination system 500 comprises 3 light sources 102A, 102B and 102C each of which is positioned on cooling platforms 104A, 104B and 104C respectively. Dichloric beam combiner 502 combines the output from light sources 102A, and 102B into a single beam which then combines with the output from light source 102C via dichloric beam combiner 504. The combined light beam exiting dichloric beam combiner 504 is fed to fiber optic cable 506. In FIG. 2C illumination system 600 comprises light sources 102A, 102B, 102C and 102D each of which is coupled to fiber optic cables 602, 604, 606 and 608 respectively.

Fiber optic cables 602, 604, 606 and 608 are all joined mechanically to a bundle or fused to a single fiber optic cable up to terminal piece 610. At some point near terminal piece 610 each one of fiber optic cables 602, 604, 606 and 608 is split into two fiber optic cables 602A&B, 604 A &B, 606A&B and 608A&B and arranged around the sclera 612 as seen in the figure.

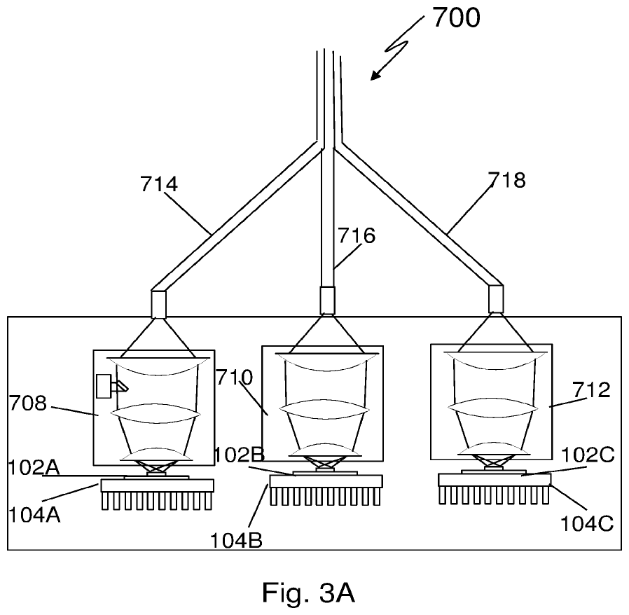

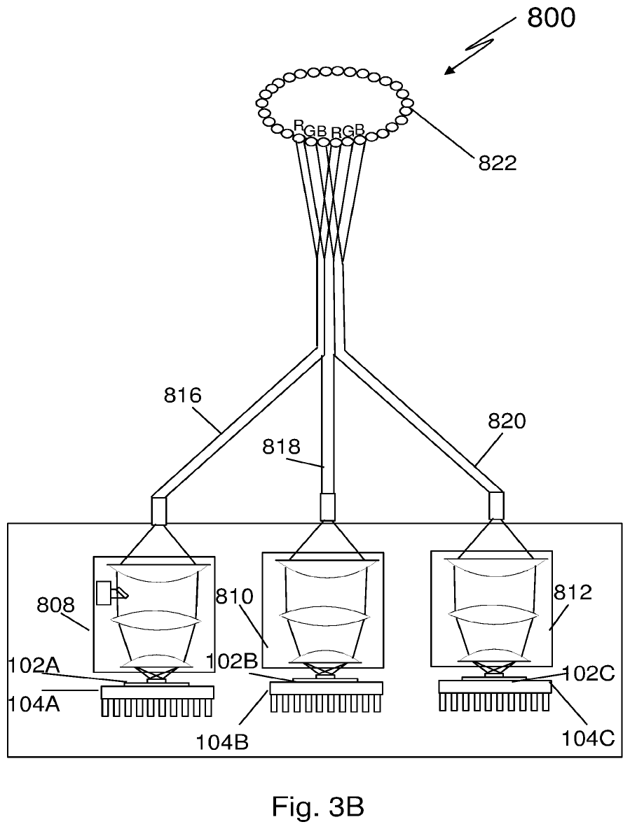

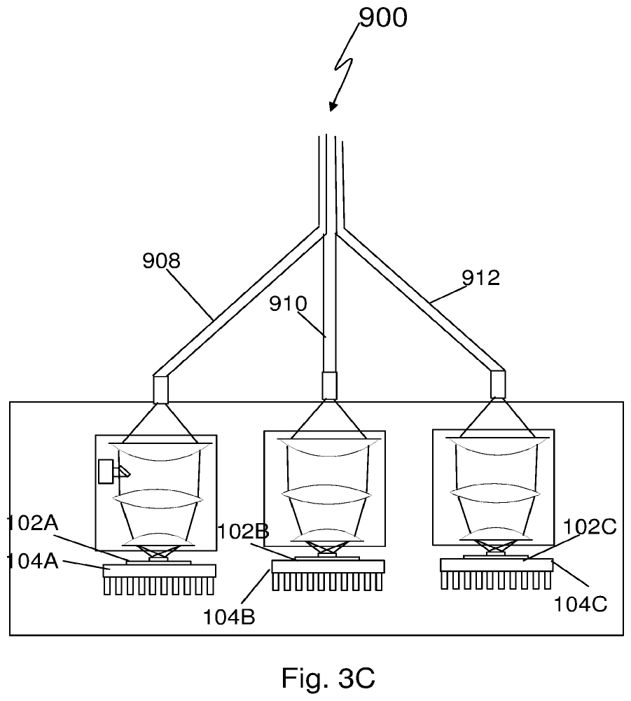

Fiber optic cables 602 A&B, 604 A&B, 606 A&B and 608 A&B contact sclera 612 at two opposing points. 4 fiber optic cables contact sclera 612 at each one of the two opposing points with red, green, blue (RGB) and NIR bands. It should be noted that other arrangements with more fiber optic cables per each color are possible as described below in FIG. 3B.

As the output from fiber optic cables 602, 604, 606 and 608 may naturally diverge, it may be necessary to add an optical system to focus, de-focus or collimate the beams as required by the application. Such an optical system may be a single or multi element system. It may be an optical element shaped to adapt the final output shape. For example, in the case of an annular fiber, the optical system may comprise a Fresnel lens with its center cut out to provide an annular lens. A flat lens, made of plastic or glass, may focus the light output to a common point as required by the application. Thus, an optical system (not shown in the figure) may be connected to fiber optic cables 602, 604, 606 and 608, to terminal piece 610, or to both.

It should be noted that fiber optic cables 602, 604, 606 and 608 are mechanically positioned in a stable manner and at the correct distance from the optical systems so that any handling of fiber optic cables 602, 604, 606 and 608 may not affect power input and output to and from the cables. Fiber optic cables 602, 604, 606 may be joined mechanically to a single bundle up to terminal piece 610 which is designed to interface with the human or animal body to provide the required diagnostic, treatment, or surgery capabilities.

It should be noted that fiber optic cables 602, 604, 606 may have various geometries other then multi strand bundles depending on the application.

It should be noted that since terminal piece 610 is in close proximity to the sclera during operation, a good coupling of the illumination light into the eye is facilitated, and due to the geometry of the terminal piece 610, illuminating all around the iris and/or between the Ora Serrata and the Equator of the eye is facilitated. Furthermore, due to the efficient coupling and scattering characteristics of the sclera, the fundus can be illuminated evenly over its entire area.

The above is true for each of the light spectral components used for such applications, i.e., blue, green, and red lights and/or near IR.

Referring now to FIG. 3A, there is shown illumination system 700 comprising light sources 102A, 102B and 102C, cooling systems 104A, 104B and 104C, optical systems 708, 710, and 712 and fiber optic cables 714, 716 and 718.

Each one of light sources 102A, 102B and 102C is coupled to each one of fiber optic cables 714, 716 and 718 via optical systems 708, 710 and 712 respectively.

Referring now to FIG. 3B, there is shown illumination system 800 comprising light sources 102A, 102B and 102C, cooling systems 104A, 104B and 104C, optical systems 808, 810, and 812 and fiber optical cables 816, 818 and 820.

Each one of light sources 102A, 102B and 102C is coupled to each one of fiber optical cables 816, 818, and 820 via optical systems 808, 810, and 812 respectively, and in this case, the various colors, red, green, and blue (RGB) are distributed in a discrete manner around the annular output 822. The color distribution as illustrated is symmetrical, however, other color arrangements are possible.

Each one of optical systems 808, 810, and 812 is positioned against each one of light sources 102A, 102B, and 102C to extract the respective output of light, to collimate the light and focus it into each one of fibers 816, 818, and 820.

Such optical systems 808, 810, and 812 may comprise multiple lenses made of glass, plastic or ceramic and having spherical, aspheric, cylindrical or any other shape. Furthermore, the optical systems 808, 810 and 812 may also include a parabolic concentrator.

Optical systems 808, 810 and 812 may be able to extract maximum power from light sources 102A-C, collimate and focus the beams to the level required by the fiber optic cables 816, 818, and 820 for best transmission/reflectance and minimum losses in the overall system.

Referring now to FIG. 3C, illumination system 900 comprising 3 light sources 102A, 1028, and 102C, cooling systems 104A, 1048 and 104C, and fiber optic cables 908, 910, and 912.

Each one of light sources 102A, 1028, and 102C is coupled to each one of fiber optic cables 908, 910 and 912 without mediating optics. Fibers 908, 910 and 912 are bundled together until reaching a terminal piece (not shown in the figure).

Illumination system 900 may be structured as follows:

Each one of light sources 102A-C is positioned on corresponding cooling systems 104A, 104B and 104C. Fiber optic cables 908, 910, and 912 are positioned close to or in contact with the emitting apertures of light source 102A-C. In this case, band pass filters and photodiodes are not needed between light sources 102A-C and fiber optic cables 908, 910, and 912 since the light sources (LEDs) emit monochromatic light. Optical systems are not needed as well in this case. Such an arrangement, called “butt coupling”, has the advantage of simple and efficient coupling.

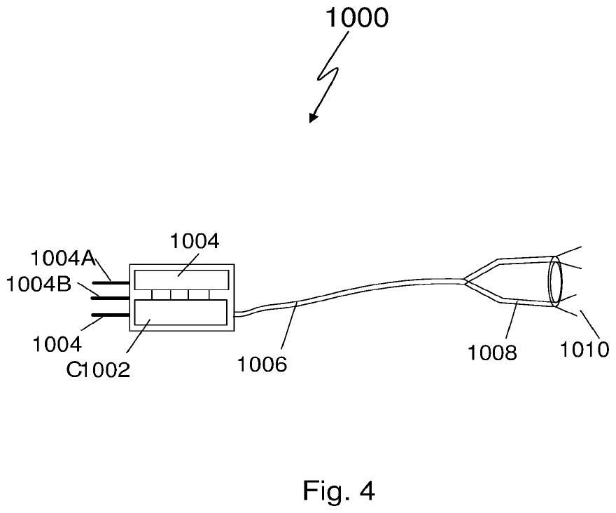

Referring now to FIG. 4, there is shown control means 1000 to control any one of the illumination systems described above. Control means 1000 comprising any one of the described light sources 1002, controller/driver 1004, and fiber optic cable 1006.

Controller/driver 1004 comprising power input 1004A, connection to central control (USB, Ethernet) 1004B, and External control lines (TTL, 24 VDC) 1004C.

Fiber optic cable 1006 is connected to annulus 1008. Annular light output 1010 is expanded from annulus 1008.

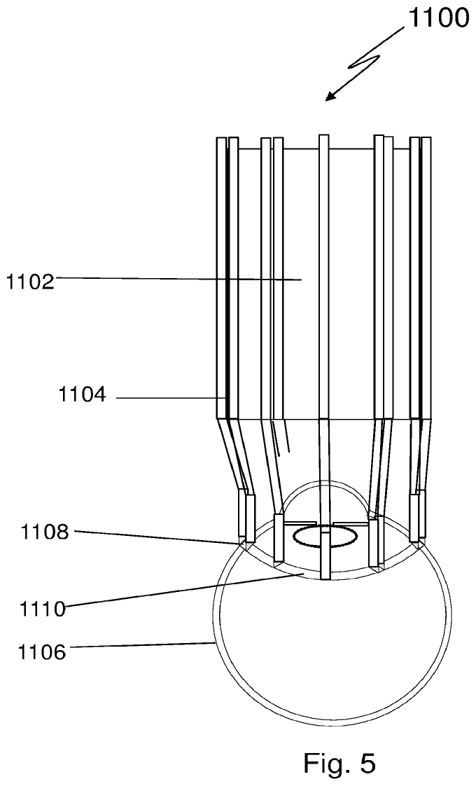

Referring now to FIG. 5, there is shown fiber optic cables distribution 1100 around the sclera. As seen in the Figure, fiber optic cables 1102, distributed around imaging lens barrel 1104, extend beyond the barrel to come in contact with the sclera 1106.

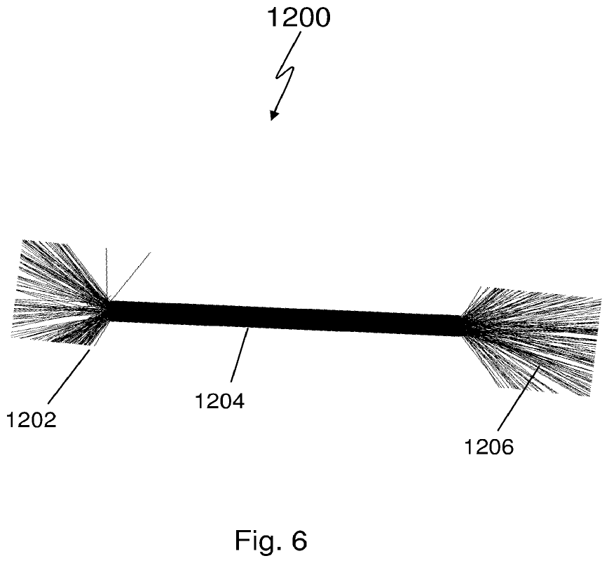

Referring now to FIG. 6, there is shown mixing rod 1200 in accordance with some embodiments of the present invention. Mixing rod 1200 having input and output cross sectional areas of 1.times.1 mm.sup.2.

Mixing rod 1200 may have square, circular, hexagonal or any other input and output cross sectional areas.

As seen in the figure, printed circuit board (PCB) with multiple-source butt 1202 enters mixing rod 1200, and mixed light 1206 is emitted in a 160-degree cone.

The light entering mixing rod 1200 travels along mixing rod 1200 in total internal reflection mode and exits mixing rod 1200 with the multiple wavelengths mixed. The degree of mixing depends on the source numerical aperture (NA), the length of mixing rod 1200 and on the geometry of the input and output surfaces of mixing rod 1200.

The output surface of mixing rod 1200 may be butt coupled to the fiber bundle input surface. The light may exit the fiber bundle homogenously, but there may still be significant losses due to NA mismatch between the output surface of mixing rod 1200 and the input surface of the fiber bundle. Light losses may be overcome by increasing system sensitivity and/or increasing light intensity.

In order to reduce light losses, a NA reducing element may be inserted between the output plane of the light source and either the fiber bundle input plane or the mixing rod 1200.

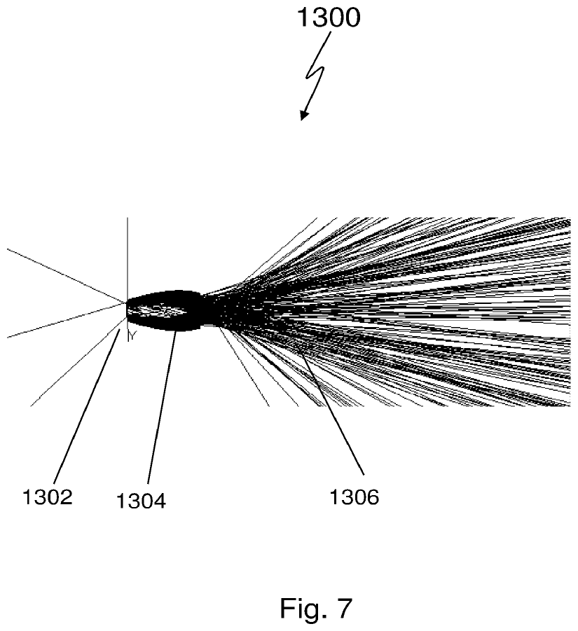

A NA reducing element may be a compound parabolic concentrator (CPC) as shown in FIG. 7 which is widely used to collimate LED strongly diverging sources.

Referring now to FIG. 7, there is shown CPC 1300 in accordance with some embodiments of the present invention. CPC 1300 having input and output cross sectional areas of 1.times.1 mm.sup.2. CPC 1300 may have parabolic, hyperbolic, conical, freeform or other cross sectional area.

Multiple-source butt 1202 enters CPC 1300, and mixed light 1302 is emitted from CPC 1300 in a 160-degree cone.

CPC 1300 may either reflect or refract the rays at high NA at an angle more compatible with fiber NA and may hardly affect the rays propagating at low NA.

Losses at CPC 1300 itself are low and mainly due to absorption or scattering.

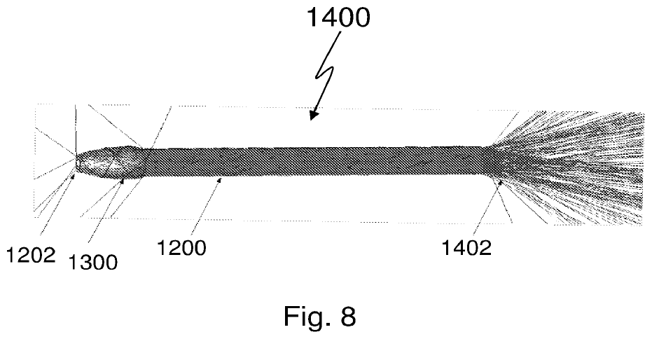

Referring now to FIG. 8, there is shown mixing device 1400 in accordance with some embodiments of the present invention. Mixing device 1400 comprising CPC 1300 connected to mixing rod 1200. Multiple-source butt 1202 enters CPC 1300, and mixed output light 1402 is emitted from mixing rod 1200 in a 160-degree cone.

In this case the output NA is significantly small, and mixed light with reduced NA is easily coupled to fiber bundle by butt coupling.

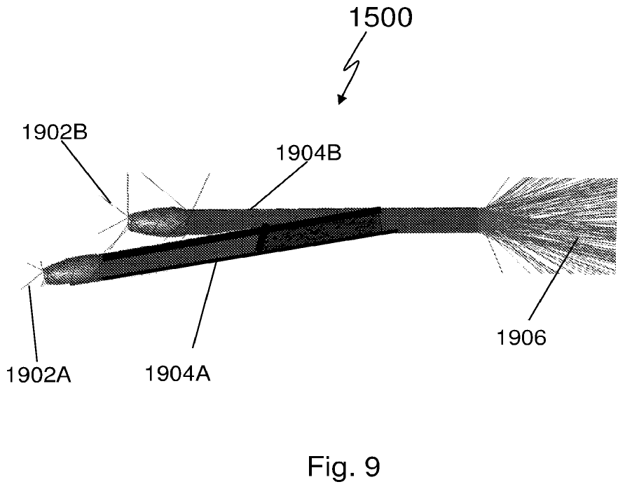

Referring now to FIG. 9, there is shown mixing device 1500 in accordance with some embodiments of the present invention. Mixing apparatus 1500 comprising mixing rod 1904A and mixing rod 1904B which are connected in a way to form a Y shaped configuration. Light sources 1902A and 1902B are fed into mixing rods 1904A and 1904B respectively. Mixed output light 1906 is emitted from mixing rod 1904B in a 160-degree cone.

It should be noted that the configuration of mixing apparatus 1500 may be expanded to include more sources in more complex geometries.

In FIGS. 6-9 multiple-source butt 1202 is mixed and coupled to a fiber optic bundle either directly or by using either mediating mixing element as mixing rod 1200 of FIG. 6, or mediating optical element as CPC 1300 of FIG. 7, or combination of both elements as mixing device 1400 of FIG. 8. In all cases the output light from the fiber bundle is characterized by a homogeneous color mixture. Thus, according to some embodiments of the present invention, an illumination system may be comprised of:

a. Light source or sources comprised of multiple wavelengths mounted close to each other either in a planar configuration or in a spherical or other configurations.

b. A compound concentrator, providing collimation capabilities, placed in close proximity to the light source/s so that the emitted light impinges on the compound concentrator.

c. A mixing rod placed in close proximity to the concentrator output enabling a homogeneous color output from the mixing rod.

d. Light guiding elements, a fiber bundle, placed in close proximity to the mixing rod output—conveying the light mixture to the useful end of the fiber bundle.

e. The end of the fiber bundle is split into individual fibers and each fiber is attached to a ring shaped structure to form a fiber annulus. The fibers are placed at an angle corresponding to sclera curvature so that when the fibers contact the sclera, they exert minimum pressure on sclera.

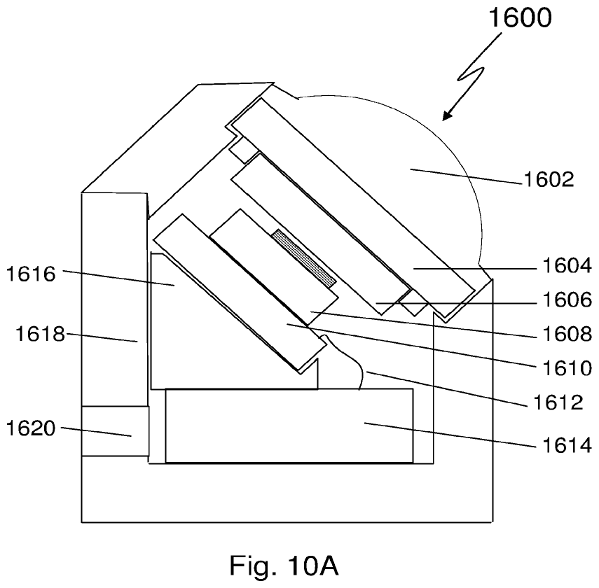

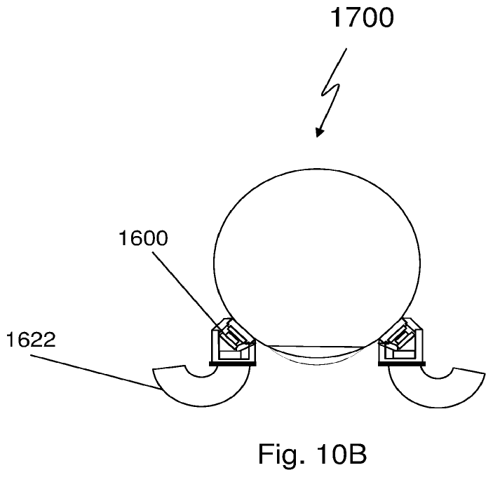

Referring now to FIGS. 10A and 10B, FIG. 10A shows a cross sectional view of LED ring 1600, and FIG. 10B shows an integrated unit 1700 comprised of LED ring 1600 of FIG. 10A and eyelid retractor 1622.

LED ring 1600 is a circumferential ring, having a tangential cross-section.

LED ring 1600 comprising disposable annular lens array 1602, fixed annular window 1604, filter per LED 1606, LED 1608, single LED PCB 1610, annular PCB 1612, wires 1614 soldered to LED PCB 1610 and to annular PCB 1612, LED PCB base 1616, ring housing 1618, and connector or cable input to annular PCB 1620.

The schematic position of LED ring 1600 on sclera is shown in FIG. 10B. As noted above, LED ring 1600 may be a stand-alone ring operated by a battery. Integrated unit 1700, in accordance with some embodiments of the present invention, may enable the use of such a battery operated LED ring as the battery may be situated in eyelid retractor 1622.

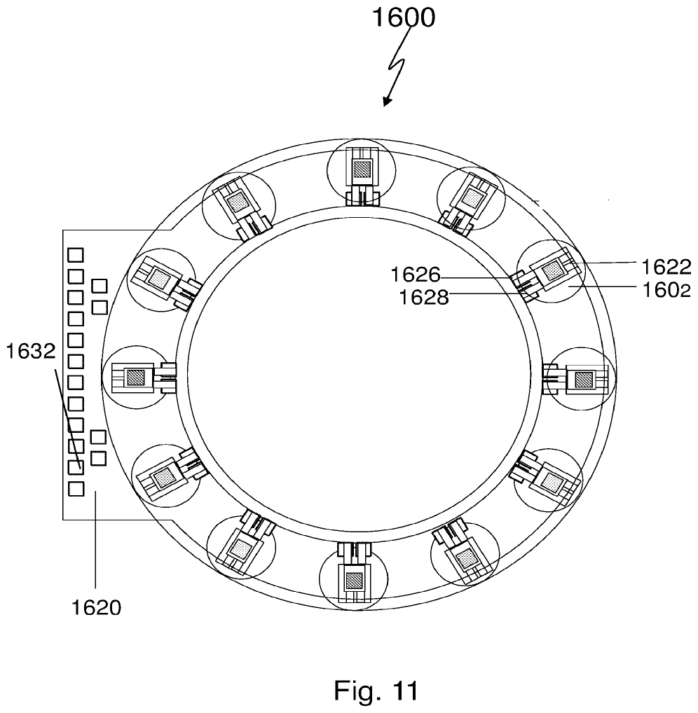

Referring now to FIG. 11, there is shown top view of LED ring 1600 with 12 LEDs-4 LEDs emitting red light, 4 LEDs emitting green light and 4 LEDs emitting blue light. Each LED 1622 comprising micro lens 1624, solder pad on PCB 1626, Be—Cu spring strip 1628, and cable or connector pads 1630.

In accordance with some embodiments of the present invention, LED ring 1600 may be placed in close proximity to the sclera with no fiber mediation. This is possible due to the miniature LEDs. For instance, the dimensions of Luxeon Z LED series from Lumiled Corporation are 1.7 mm.times.1.3 mm.times.0.7 mm with a 1 mm.times.1 mm emitter. Such dimensions enable placing up to about 18 LEDs in LED ring 1600 and around the eye globe with the LEDs pointing at the ora serrata for best transmission through the sclera and through the pars plana zone up to the eye equator.

In accordance with some embodiments of the present invention, LEDs of various wavelengths may be placed around ring 1600, and there may be an equal number of LEDs emitting light of same color around ring 1600. For instance, a 4 color ring may be assembled with 16 LEDs where 4 LEDs emitting same color are placed in a cross configuration. Such an arrangement ensures equal illumination of the whole fundus with each color.

In other configurations RGB LEDs may be placed around ring 1600 in asymmetrical geometries. For example, two sets of RGB LEDs may be placed around ring 1600 with 180 degrees with respect to each other or any other geometric arrangement required for efficient illumination of the retina.

In other configurations, the ring may comprise light sources of a single wavelength for providing greater illumination at that wavelength. For example, when performing angiography, the ring may consist a single or multiple LEDs operating only at the required excitation wavelength.

In accordance with some embodiments of the present invention, each one of the LEDs is soldered to an individual PCB 1632 and placed on ring 1600. In this case, ring 1600 is designed to hold the LEDs in place where the light outputs are aimed in a direction perpendicular to the sclera.

Ring 1600 is placed on annular PCB 1632 where each LED is connected with two wires to the annular PCB 1632. The annular PCB 1632 may operate the LEDs in series or in parallel and may enable separate control of each LED or group of LEDs. The annular PCB 1632 has a connector or solder pads for cable connection.

In another configuration, LED PCBs are wired together since there is no annular PCB 1632. However, this configuration is less convenient due to wiring complexities and wires volume.

In yet another configuration each LED 1622 is connected to the annular PCB 1632 by two Be—Cu leaf spring 1628 with no LED PCB mediation. The two springs act as current conductors and heat conductors.

The supporting area of ring 1600 in contact with the LED PCB and the overall supporting structure may warm up, and ring 1600 may be cooled down by conduction to the surrounding air.

If filtering is needed, a band pass filter 1606 may be placed after each LED 1608.

The fixed annular window 1604 is the part of ring 1600 that contacting the sclera. Fixed annular window 1604 may include a micro lens 1602 per LED for collimating the LED's light. Such micro lens 1602 directing a greater amount of the LED light into the eye instead of losing the light to ring light interior or in other directions.

Window 1604 is designed to adapt to the curvature of the sclera, and the design may be adapted to eye dimensions of neonatal, adults and animals.

Ring 1600 and annular PCB 1632 structure are enclosed by a plastic and/or metal structure.

All parts of ring 1600 may be either printed (including plastic optics) or manufactured by conventional machining processes.

In a different configuration, the fixed annular window 1604 may be made from 2 parts: a fixed part and a disposable part. The fixed part is a window, the disposable part may be either a window, a micro lens, a silicon cover or any other material conforming with medical regulatory acceptance. The disposable part is the only part that comes in actual contact with the eye.

After each examination the disposable part is easily removed and disposed. A new disposable part is easily inserted making the unit ready for another test. Thus, sterilization of the fixed part is not required.

It should be noted that the structure of ring 1600 may warm up only slightly and may not reach a temperature that may be hazardous for the following reasons:

a. Light source/s of each color is/are operated separately and for a short duration.

b. Light source is in close proximity to the eye, thus transmission losses are minimal—the operation current may be low and consequently heat generation may be low as well.

c. Fixed annular window is in touch with the sclera and is thermally isolated from the support structure.

d. Temperature detection element, such as a thermocouple, may be connected to the ring and if the temperature reaches the safety limit, the control system may disconnect the current.

It should be noted that in accordance with some embodiments of the present invention, each LED may be controlled by a central control system or a system computer. Optionally a common controller may control all LEDS and may be connected to a central control system or system computer.

In accordance with the present invention, the controller monitors electrical power injected to each LED and may monitor the optical output of each LED. The controller incorporates all necessary safety features to ensure correct and safe operation of the illumination system.

In accordance with some embodiments of the present invention, the illumination system may be either manually controlled (using keyboard or switches on unit), voice activated or even activated via pedals.

In accordance with the present invention, packaging the illumination system enables safe and secure positioning of the light sources, optics, filters and cables. The packaging contains a cover to protect users from possible scattered light and to enable cable connection.

### EXAMPLES

### Example I—Fundus Imaging

The illumination system, in accordance with the present invention, enables efficient, homogeneous and safe illumination of the fundus at one or more colors typically ranging from 440 nm to about 800 nm. The fundus can be photographed using one color providing an image of features illuminated at that color. In accordance with the present invention, images can be acquired at three colors such as blue, green and red and the images computer combined to provide a full true color image. Any combination of colors can be used to provide specific details to a required diagnosis. Illumination using the disclosed invention requires no moving parts and images can be taken at different colors very rapidly using the fast modulation characteristics of the SSLS.

### Example II—Fluorescein Angiography

Fluorescein angiography is a technique for examining the circulation of the retina and choroid using a fluorescent dye and a specialized camera. It involves injection of sodium fluorescein into the systemic circulation, and then an angiogram is obtained by photographing the fluorescence emitted after illumination of the retina with blue light at a wavelength range of 490-520 nanometers. The disclosed invention enables homogeneous illumination at the required wavelength.

A separate imaging system monitors the emission from the fluorescein.

### Example III—ICG Angiography

Indocyanine Green angiography (ICG) is a procedure which images the choroid. This layer, the choroid, is deeper than the retina and normally obscured by pigmentation. In contrast with sodium fluorescein, ICG fluoresces in the infrared after excitation at around 800 nm. The disclosed invention enables homogeneous illumination at the required wavelength using an IR LED or an IR diode laser. A separate imaging system monitors the emission from the ICG.

What is claimed is:

1. An integrated ophthalmic illumination system comprising:
a circumferential ring, having a tangential cross-section;
at least one light source comprising a light beam of multiple wavelengths;
a mediating mixing element, said mediating mixing element placed in proximity to said at least one light source to receive and to transform said light beam into a mixed beam;
a plurality of light guiding elements, said plurality of light guiding elements placed in close proximity to the output of said mediating mixing element to receive and to convey said mixed beam to said circumferential ring;
and a controller connected to said at least one light source for controlling light intensity, light distribution, and restricted light of predetermined wavelengths.

2. The system according of claim 1, wherein the light source is a solid state light source selected from the group consisting of: LEDs, diode lasers, and diode pumped solid state lasers.

3. The system according of claim 1, wherein said controller enabling separate control of each one of the plurality of light sources.

4. The system of claim 1, wherein said controller monitors electrical power injected to each one of said plurality of light sources.

5. The system of claim 1, wherein said controller monitors the optical output of each one of said plurality of light sources.

6. The system of claim 1, wherein said illumination system is activated either via voice, pedals or manually.

7. The system of claim 1, wherein said mediating mixing element comprises a compound concentrator.

8. The system of claim 1, wherein said mediating mixing element comprises at least one mixing rod.

9. The system of claim 1, wherein said mediating mixing element comprises two mixing rods forming a Y-shaped configuration.

10. The system of claim 1, wherein said mediating mixing element comprises a compound concentrator and at least one mixing rod.

11. The system of claim 1, wherein said mediating mixing element comprises a single optical fiber.

12. The system of claim 1, wherein said mediating mixing element comprises a plurality of optical fibers.

13. The system of claim 1, wherein said mediating mixing element comprises at least one optical fiber bundle.
